(19) United States
(12) Patent Application Publication
(10) Pub. No.: US 2006/0252727 A1
(43) Pub. Date: Nov. 9, 2006

(54) Medicinal compositions and therapeutic methods for treating arthritis and other painful conditions

(76) Inventors: Seymour Ehrenpreis, Skokie, IL (US); Eli D. Ehrenpreis, Skokie, IL (US)

Correspondence Address: JONATHAN D. FEUCHTWANG, 1330 CENTRAL AVENUE, DEERFIELD, IL, 60015, US

(21) Appl. No.: 11/479,216

(22) Filed: Jun. 30, 2006

Related U.S. Application Data
(63) Continuation of application No. 10/497,631, filed on Jun. 3, 2004.
(60) Provisional application No. 60/338,320, filed on Dec. 6, 2001.

(51) Int. Cl. A61K 31/737; A61K 31/7008; A61K 31/198; A61K 31/192

(52) U.S. Cl. 514/54; 514/62; 514/567; 514/561; 514/570

(57) ABSTRACT

The compositions of this invention comprise a mixture of (1) phenylanine and a dietary food supplement, (2) leucine and a dietary food supplement, and (3) hydrocinnamic acid and a dietary food supplement. The compositions are used for medicinal purposes, to alleviate acute and chronic arthritis and other painful conditions.

### RELATED PATENT APPLICATIONS & INCORPORATION BY REFERENCE

This application is a continuation-in-part of U.S. application Ser. No. 10/497,631 filed Jun. 3, 2004 which claims priority from PCT application US02/38898 filed Dec. 5, 2002 which claims priority from U.S. provisional patent application Ser. No. 60/338,320, entitled “Anti-Inflammation/Analgesic Compositions & Methods Of treating Arthritis And Other Painful Conditions,” filed Dec. 6, 2001. This related application is incorporated herein by reference and made a part of this application. If any conflict arises between the disclosure of the invention in this application and that in the related provisional application, the disclosure in this application shall govern. Moreover, Applicants incorporate herein by reference any and all U.S. patents, U.S. patent applications, and other documents cited or referred to in this application or cited or referred to in the U.S. patents and U.S. patent applications incorporated herein by reference.

### DEFINITIONS

The words “comprising,” “having,” and “including,” and other forms thereof, are intended to be equivalent in meaning and be open ended in that an item or items following any one of these words is not meant to be an exhaustive listing of such item or items, or meant to be limited to only the listed item or items.

The term “arthritis” means inflammation of the articular extremity of a bone resulting in erosion of the cartilage, loss of range of motion and pain.

### BACKGROUND OF INVENTION

The amino acid phenylalanine is now shown by the inventor to be a medication for treating various maladies, including arthritis. This medication includes D-phenylalanine (DPA) or D, L-phenylalanine (DLPA); the latter is a 50-50 mixture of the D-(DPA) and L-(DLPA) forms of phenylalanine. Phenylalanine is considered to be a dietary supplement; DLPA is sold over-the-counter. DPA, but not LPA, is an inhibitor of enzymes that inactivate the naturally-occurring morphine-like peptides, termed enkephalins. By this action, DPA promotes the accumulation of the enkephalins in the central nervous system and elsewhere in the body, e.g., joints. Thus it is the DPA not LPA which is the active agent in providing relief in arthritis and other painful conditions.

DPA has been shown to have another activity, namely, as an anti-inflammatory agent. These activities—analgesia plus anti-inflammatory activity—suggest efficacy of DPA or DLPA in arthritis; data indicating that this is indeed the case is presented below.

Other substances having the same activities as DPA include D-leucine, D, L-leucine and hydrocinnamic acid.

In general, the cause of arthritis is not known, particularly, osteoarthritis. Rheumatoid arthritis is an autoimmune disease. Osteoarthritis in animals and humans is attributed to excessive amounts of naturally-occurring inflammatory and pain-producing factors such as prostaglandins and other products of the arachidonic acid cascade. In addition, destructive proteolytic enzymes are elaborated during the disease process. Conventional treatment of arthritis consists of drugs which attempt to prevent the accumulation of these deleterious pathophysiological factors. It is now proposed to treat arthritis and various painful conditions in animals and humans by raising the levels of endorphins in various regions of the body. A primary agent in the new approach consists of a combination of drugs and natural products along with DPA in doses sufficient to reverse or prevent the inflammation, pain and tissue destruction that occurs during arthritis.

The sequence of events that occurs in an arthritic joint is as follows. If a pathophysiological event occurs, e.g. trauma, joint infection, autoimmune response, the arachidonic acid cascade is activated, resulting in the generation of prostaglandins and leukotrienes; these substances promote inflammation and pain. Cell membranes within the joint are damaged, resulting in the release of proteolytic enzymes, which can erode the cartilage. Also present within joint synovial fluid are various endorphins (enkephalins, beta endorphin); these are analgesic peptides. Under ordinary conditions, they would counteract the pain caused by the products of the arachidonic acid cascade. However, if the conditions persist, or are particularly severe, the condition becomes irreversible and full-blown arthritis ensues.

The reasons for this are as follows. Under such circumstances, the amount of proteolytic enzymes released becomes sufficient to degrade the joint cartilage, causing its destruction. In addition, these enzymes degrade the endorphins present. Finally, there is a build-up of the products of the arachidonic acid cascade. The net result is continuing pain and inflammation and loss of range of motion, i.e., the arthritic condition.

There are several classes of drugs which, to a certain extent, can alleviate the painful condition as well as the inflammatory response. These include the following: various steroids, aspirin and other non-steroidal anti-inflammatory drugs (NSADs) such as ibuprofen, indomethacin, naprosyn, sulindac, etc., and more recently, the COX₂ inhibitors, Vioxx and Celebrex. These drugs block the arachidonic acid cascade, thereby providing relief from the inflammation and pain caused by the prostaglandins and related pathophysiological compounds within the joint.

In addition, there are drugs which may slow down the progress of the disease; these are called DMARDs (disease modifying anti-arthritic drugs) and include methotrexate, cyclosporine, and various gold compounds. Unfortunately, all of these drugs have their limitations, in that they may cause serious, even fatal, adverse reactions. In addition, these drugs are involved with many interactions with other drugs; such interactions, at times, may be very deleterious, requiring difficult adjustment of dosage for the interacting drugs.

More recently, several nutraceuticals have been introduced with some success for arthritis treatment. Among these are glucosamine (GS) and chondroitin sulfate (CSA). These naturally-occurring compounds offer a new approach to treatment, namely, to slow down, or perhaps even reverse the destruction of joint cartilage that is characteristic of arthritis. It should be noted that none of these newer agents have analgesic or anti-inflammatory activity and have a very long onset of action.

### SUMMARY OF INVENTION

This invention, with its several desirable features, is summarized in the CLAIMS that follow. After reading the following section entitled “DETAILED DESCRIPTION OF SOME EMBODIMENTS OF THIS INVENTION,” one will understand how the features of this invention provide its benefits. The benefits of this invention include, but are not limited to, treating the following: preventing or reversing the inflammation and chronic acute pain of headache, toothache, back pain, musculoskeletal pain, pre-menstrual pain, pain due to sports injury, post-operative surgery pain, and dental pain.

The compositions of this invention comprise:
(1) a mixture of DPA or DLPA and one or more of the dietary food supplements identified below;
(2) a mixture of D-leucine or D, L-leucine and one or more of the dietary food supplement identified below; and
(3) a mixture of hydrocinnamic acid and one or more of the dietary food supplement identified below;
(4) a mixture of DPA or DLPA, D-leucine or D, L-leucine and one or more of the dietary food supplement identified below;

The D-phenylalanine, D-leucine, hydrocinnamic, or mixtures thereof, may be present in an amount of from 5 to 95 weight percent and the dietary food supplement may be present in an amount of from 5 to 95 weight percent. The phenylalanine may include as the major component the D-phenylalanine, and the leucine may include as the major component the D-leucine.

The dietary food supplements consist of glucosamine, chondroitin sulfate, or a mixture of white willow bark powder and extract of salicin and a number of others listed below.

The various individual combinations of the above ingredients are each unique and some combinations are more suited to treating specific maladies than others.

In one embodiment of this invention for treating arthritis, a therapeutic amount of the primary agent DPA or DLPA is blended with a therapeutic amount of one or more nutraceuticals, e.g., glucosamine, glucosamine plus chondroitin sulfate, ginger evening primrose, stinging nettle, white willow bark, borage, Devil's claw, bromelain (or pineapple extract) and turmeric. Each of these nutraceuticals has been shown to be effective for treating arthritis.

In a second embodiment of this invention for treating arthritis, the DPA or DLPA is blended with a therapeutic amount of a non-steroidal, anti-inflammatory drug (hereinafter “NSAID”) or COX₂ inhibitor (an NSAID) along with one or more of an appropriate nutraceutical.

In a third embodiment of this invention for treating arthritis, a therapeutic amount of D-leucine, D, L-leucine or hydrocinnamic acid may be blended with one or more nutraceuticals, e.g., glucosamine, glucosamine plus chondroitin sulfate, etc.

In a fourth embodiment of this invention for treating arthritis, the D-Leucine, D, L-leucine or hydrocinnamic acid is blended along with a therapeutic amount of an NSAID.

In a fifth embodiment of this invention for treating non-arthritic pain, a therapeutic amount of DPA, DLPA, D-leucine, D, L-leucine or cinnamic acid is combined with a therapeutic amount of an NSAID plus a therapeutic amount of a nutraceutical such as ginger, evening primrose, stinging nettle, white willow bark, borage, Devil's claw, bromelain and turmeric. Each of these nutraceuticals has been shown to be effective for treating many painful conditions.

In these embodiments, the amount of DPA administered is between approximately 10 and approximately 3000 mg/day, and the amount of DLPA administered is between approximately 20 and approximately 6000 mg/day. Administration may be in the form of a tablet or a capsule.

Ginger, evening primrose, stinging nettle and white willow bark, borage, Devil's claw, nettle, bromelain, and turmeric are nutraceuticals shown to be effective for treating arthritis and other painful conditions. Their use with DPA, DLPA, D-leucine, D, L-leucine, hydrocinnamic acid, or NSAIDs could provide enhanced efficacy for treating those who do not respond well to other treatments.

Each of the components of the proposed combinations provides some advantages over individual usage, particularly for treatment of arthritis. DPA/DLPA provides relief of pain, as do the NSAIDs. The combination of NSAIDs with DPA or DLPA has been shown to greatly enhance the analgesia provided by the NSAIDs alone. Thus, the combination provides pain relief for those who fail to respond, or respond inadequately, to DPA or DLPA alone or to NSAIDs alone. DPA/DLPA combined with the NSAIDs has potent anti-inflammatory activities. DPA/DLPA is extremely safe as shown both in animal and human studies. Glucosamine and chondroitin sulfate can promote regeneration of cartilage, which may be destroyed in arthritis. Thus, the combinations discussed above provide the following advantages over their individual use:

1. Greater efficacy for relief of the pain of arthritis and other painful conditions.

2. Reduced drug toxicity, in particular, by enabling reduction of dosage of acetaminophen, aspirin and other NSAIDs used for treating arthritis and other painful conditions.

3. Reduced incidence of drug-drug interactions due to the reduction in dosage of Aspirin or other NSAIDs used for treatment of arthritis and other painful conditions.

4. Possibility of reversing inflammation that accompanies arthritis.

5. Possibility of stopping cartilage destruction and rebuilding cartilage destroyed by the arthrititic processes, through the use of glucosamine/chondroitin sulfate, in combination with DPA and/or NSAIDs.

6. Providing more rapid relief from pain and inflammation by using DPA or DLPA plus NSAIDs in conjunction with glucosamine/chondroitin sulfate, rather than the latter combination alone.

One aspect of the present invention is to use DPA, DLPA, D-leucine, D, L-leucine or hydrocinnamic acid, or combinations of these ingredients, to inhibit the enzymes that inactivate certain endorphins, in particular, the enkephalins, thereby permitting these endorphins to accumulate in the central nervous system and other regions of the body, e.g., synovial fluid of joints. In so doing, these endorphins relieve the symptoms of pain and/or inflammation, where it is present in arthritis and other painful conditions. The compositions of this invention may include one or more NSAIDs known to be useful for treating such conditions.

All of the compounds and combinations described above can be offered as pharmaceutically-accepted formulations, using methods known to those with ordinary skill in the art. These formulations may only be administered by the oral route, whether solely or in combination. The dosage of DPA/DLPA, D-leucine or hydrocinnamic acid, or combinations of these ingredients, or in combination with any of the other compounds, will depend on the severity of the arthritic, or other painful condition, as well as any existing or potential co-morbidity. It should be noted that the present invention has application for both human and veterinary use. Suggested oral dosages for humans are shown in Table 5. Formulations for human use will be in the form of tablets or capsules, normal and sustained release.

### BRIEF DESCRIPTION OF THE TABLES

Table 1: Anti-inflammatory activity of DPA using the rat paw carrageenan method.

Table 2: Anti-inflammatory activity of DPA using the mouse writhing test.

Table 3: Analgesic activity of DPA using the mouse hot plate method. Combination of DPA with indomethacin and sulindac, two NSAIDs.

Table 4: Efficacy of DPA in arthritis and a variety of other musculoskeletal diseases: Human studies.

Table 5: Suggested dosage schedule for treating arthritis in humans using DPA or DLPA alone or in combination with various other compounds.

Table 6: List of NSAIDs, including COX₂ Inhibitors

Table 7: List of dietary food supplements combined with DPA.

### DETAILED DESCRIPTION OF THE METHODS CITED ABOVE AND RESULTS OBTAINED

Anti-inflammatory activity of DPA using the rat paw carrageenan method. This is a generally recognized method for evaluating anti-inflammatory drugs of the aspirin/NSAID (as used throughout this application NSAID refers to a non-steroidal, anti-inflammatory drug) type or those that counteract the action of the prostaglandins. Such drugs are useful for treating conditions such as arthritis in animals and humans. In this method, the paw of the rat is injected with carrageenan, which causes swelling of the paw due to the accumulation of fluid. A drug that prevents this swelling would be expected to have anti-inflammatory and hence, anti-arthritic activity in humans. In this study, DPA was administered by various routes and times into groups of rats (6 male albino rats per group-weight approximately 250 grams) before carrageenan and the degree of swelling was measured by the extent of displacement of water when the paw was immersed into a calibrated cylinder. A placebo consisting of saline solution was similarly administered.

TABLE 1
Anti-inflammatory activity of DPA using the rat paw carrageenan
method

%
Inhibition
of swelling

A. Subcutaneous injection

| | | | | |
|---|---|---|---|---|
| 120 minutes | Placebo | | | 0 |
| before | DPA | 8 | mg/kg | 15 |
| carrageenan | | 125 | mg/kg | 37 |
| | | 500 | mg/kg | 35 |
| 6 hours | Placebo | | | 0 |
| Before | DPA | 62.5 | | 35 |
| Carrageenan | | 250 | | 36 |
| | | 500 | | 75 |

B. Oral administration

| | | | | |
|---|---|---|---|---|
| 150 minutes | Placebo | | | 0 |
| before | DPA | 125 | mg/kg | 7 |
| carrageenan | | 250 | mg/kg | 10 |
| | | 500 | mg/kg | 24 |
| | | 1000 | mg/kg | 62 |
| | | 1500 | mg/kg | 79 |

Anti-inflammatory activity of DPA using the mouse phenylbenzylquinone (PBQ) writhing test. This is a generally recognized method for evaluating anti-inflammatory drugs of the aspirin/NSAID type, i.e. those drugs which counteract the action of the prostaglandins. Such drugs are useful for treating conditions such as arthritis in animals and humans.

In this method, a solution of PBQ was injected subcutaneously into groups of 6 mice (male, albino, 22-25 grams). Within a few minutes, the mice began to writhe. DPA was injected intra-peritoneally or given by mouth at different times prior to PBQ. The number of writhes was counted over a period of 20 minutes. The effects of DPA were compared to that of similarly administered saline control. Results are shown in Table 2. A drug that blocks writhing would be expected to have anti-inflammatory and hence anti-arthritic activity in humans.

TABLE 2
Anti-inflammatory activity of DPA using the mouse writhing test

| | |
|---|---|
| | % inhibition of |
| Number of writhes | writhing |

A. Subcutaneous injection, 6 hours before PBQ

| | | | | |
|---|---|---|---|---|
| Saline | | | 110 | — |
| DPA, | 500 | mg/kg | 28 | 75 |
| Saline | | | 166 | — |
| DPA, | 250 | mg/kg | 106 | 36 |
| | 62.5 | mg/kg | 108 | 35 |

B. Oral administration of DPA, 2.5 hours before PBQ

| | | | | |
|---|---|---|---|---|
| Saline | | | 148 | — |
| DPA, | 250 | mg/kg | 133 | 10 |
| | 500 | mg/kg | 110 | 24 |
| | 1000 | mg/kg | 56 | 62 |

C. Intra-peritoneal injection of DPA, 2 hours before PBQ

| | | | | |
|---|---|---|---|---|
| Saline | | | 125 | — |
| DPA, | 250 | mg/kg | 34 | 73 |
| | 500 | mg/kg | 13 | 90 |

Analgesic activity of DPA using the mouse hot plate test. The mouse hot plate is a generally recognized method for evaluating analgesic drugs that act on the endorphin system, e.g., morphine-like drugs and drugs that increase endorphin levels, e.g., DPA. Such drugs are useful for treating many kinds of painful conditions, including arthritis, headache, low back pain, musculoskeletal pain, pre-menstrual pain, and dental pain.

In this method, groups of mice (6 albino mice per group, weighing between 22 and 25 grams) are individually placed on a hot plate at 55° C. and a beaker is placed over the mouse. The time for the mouse to jump in an attempt to escape the painful condition is recorded. An analgesic substance is one which increases the threshold to pain, thereby permitting the mouse to remain on the hot plate for longer times. The effect of the drug is compared to that of a saline control. DPA at different doses was injected intra-peritoneally into the mice 2 hours before they were placed on the hot plate, and jumping time determined over the next 1-2 hours. The effect of DPA was compared with that of an injection of saline.

For studies involving combinations of drugs, the combination was injected at the same time. In the studies shown below, the second drug consisted of indomethacin, diclofenac, or acetaminophen. Each of these drugs is used for treating arthritis, as well as many other painful conditions.

TABLE 3
Analgesic activity of DPA, DPA plus indomethacin, DPA plus
diclofenac and DPA plus acetaminophen, using the mouse
hot plate method

| | Increase in the |
|---|---|
| | threshold to |
| | jumping, compared |
| | to control |
| DPA, 250 mg/kg | 3-fold |
| Indomethacin, 20 mg/kg | 0 |
| DPA, 250 mg/kg PLUS indomethacin, | 11-fold |
| 20 mg/kg | |
| Diclofenac, 20 mg/kg | 0 |
| DPA, 250 mg/kg PLUS diclofenac, 20 mg/kg | 12-fold |

As shown in Table 3, DPA increased the threshold time to jump by three-fold, as compared to saline control. As expected, the NSAID was ineffective in increasing the pain threshold by this method. The data showed that the combination of DPA with either indomethacin or diclofenac provided a greatly enhanced degree of analgesia, far greater than the sum of the individual components of the combination. Similar results were obtained when DPA was combined with acetaminophen (Tylenol).

4. Efficacy of DPA treatment for arthritis and a variety of other musculoskeletal diseases; human studies. Forty-three patients were studied in an open-label, cross-over procedure. Of these, 32 had arthritis. All of the patients had previously been given many treatments, without success, in relieving their pain. Pre-DPA administration treatments used included such potent drugs as Percodan, Darvocet, Valium, and aspirin, as well as acupuncture. Prior to giving DPA 800 mg/day by mouth, all treatments were stopped. Patients were given a card on which they scored their degree of pain, on a scale of 1-4; 1 indicated no relief, 2 indicated approximately 25% relief, 3 indicated approximately 50% relief, and 4 indicated complete relief. Results are shown in Table 4.

TABLE 4
Efficacy of DPA in humans with arthritis and other skeletal diseases

| Complete relief | Excellent relief of | Moderate relief | No relief of |
|---|---|---|---|
| of pain | pain | of pain | pain |
| 2 | 7 | 19 | 15* |

*Most of these patients took DPA for only 1-2 weeks. They discontinued the DPA because of a lack of success. For very severe conditions, DPA should be taken for longer periods of time and at even higher doses.

What these human results show is that DPA alone, at the moderate dose of 800 mg/day could successfully decrease one of the prominent signs of these diseases, namely pain.

Pain is perhaps the most debilitating aspect of arthritis. In this invention, we propose to use DPA or DLPA, along with other drugs or natural substances, in order to enhance the analgesic efficacy of DPA in arthritis. Table 5, 6 and 7 presents some suggested dosages for using DPA, or DLPA alone, or in combination with the various compounds discussed previously for treating arthritis and a variety of painful conditions in humans.

TABLE 5
Suggested dosage schedule for treating arthritis in humans, using DPA,
DLPA alone or in combination with glucosamine chondroitin sulfate.

| | | Dose of second |
|---|---|---|
| Dose of DPA | Dose of DLPA | component |
| 10-3000 mg/day | — | — |
| — | 20-6000 mg/day | — |
| 10-3000 mg/day | — | Glucosamine: 1000-1500 mg/ |
| | | day |
| — | 20-6000 mg/day | Glucosamine: 1000-1500 mg/ |
| | | day |
| 10-3000 mg/day | — | Chondroitin sulfate: |
| | | 100-1000 mg/day |
| — | 20-6000 mg/day | Chondroitin Sulfate: |
| | | 100-1000 mg/day |

Similar combinations of DPA or DLPA with all other NSAIDs are herein proposed, including the new COX 2 inhibitors. (See Table 6)

TABLE 6
NSAIDs, including COX₂ Inhibitors which may be
combined with DPA, DLPA, D-Leucine, hydrocinnamic acid or
the nutraceuticals listed in Table 7.

| | Approximate doses, |
|---|---|
| Name | mg/day |
| Aspirin | 85-3000 |
| diclofenac | 25-300 |
| Etadolac | 100-1500 |
| fenoprofen | 300-4000 |
| fluriprofen | 100-600 |
| ibuprofen | 200-6000 |
| indomethacin | 10-400 |
| ketoprofen | 50-400 |
| ketorolac | 10-120 |
| meclofenamate | 100-800 |
| nabumetone | 250-4000 |
| naproxen | 100-3000 |
| oxaprozin | 600-3000 |
| piroxicam | 5-40 |
| sulindac | 50-1000 |
| tolmetin | 400-4000 |
| rofecoxib | 10-150 |
| celecoxib | 100-400 |

TABLE 7
Nutraceuticals to be combined with DPA, DLPA, D-leucine, D, L-
leucine, hydrocinnamic acid, or an NSAID; approximate doses.

| | Approximate doses, | |
|---|---|---|
| Name | mg/day | |
| borage oil | 1.1 or 0.4 | grams |
| devil's claw (root) | 4.5 | grams |
| ginger (root) | 1,000 | mg. |
| evening primrose oil | 540 mg-2.8 g. | |
| nettle (above-ground parts) | ¼ tsp of tincture, 1 to 2 | |
| | times per day | |
| turmeric | 400-600 | mg. |
| willow (dry bark) | 100-500 | mg. |
| bromelain | 300-1500 | mg. |
| white willow powder and | 100-500 | mg. |
| extract 0.05-.99% salacin | | |
| Type II collagen | 100-2400 | mg. |
| methylysulfonyl-methane | 500-2500 | mg. |
| (MSM) | | |

The above presents a description of the best mode contemplated of carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above which are fully equivalent. Consequently, it is not the intention to limit this invention to the particular embodiments disclosed. On the contrary, the intention is to cover all modifications and alternate constructions coming within the spirit and scope of the invention, as generally expressed by the following claims, which particularly and distinctly claim the subject matter of the invention:

1. A composition comprising a therapeutic dose of D-phenylalanine (DPA) 50 mg to 5 grams or D,L-phenylalanine (DLPA) 100 mg-10 grams for the treatment of acute or chronic arthritis and other painful conditions.

2. A composition as in claim 1 plus a dietary amount of glucosamine.

3. A composition as in claim 1 plus a dietary amount of chondroitin sulfate.

4. A composition as in claim 2 plus a dietary amount of glucosamine plus chondroitin sulfate.

5. A composition as in claim 2 plus a therapeutic amount of a non-steroidal, anti-inflammatory drug.

6. A composition as in claim 3 plus a therapeutic amount of a non-steroidal, anti-inflammatory drug.

7. A composition as in claim 4 plus a therapeutic amount of a non-steroidal, anti-inflammatory drug.

8. A composition comprising a therapeutic dose of D-leucine (50 mg to 5 grams) or D, L-leucine (100 mg to 5 grams) for the treatment of acute or chronic arthritis.

9. A composition as in claim 8 plus a dietary amount of glucosamine.

10. A composition as in claim 8 plus a dietary amount of chondroitin sulfate.

11. A composition as in claim 8 plus a dietary amount of glucosamine plus chondroitin sulfate.

12. A composition as in claim 9 plus a therapeutic amount of a non-steroidal, anti-inflammatory drug.

13. A composition as in claim 10 plus a therapeutic amount of a non-steroidal, anti-inflammatory drug.

14. A composition as in claim 11 plus a therapeutic amount of a non-steroidal, anti-inflammatory drug.

15. A composition comprising a therapeutic dose of hydrocinnamic acid (50 mg to 5 grams) for the treatment of acute or chronic arthritis.

16. A composition as in claim 15 plus a dietary amount of glucosamine.

17. A composition as in claim 15 plus a dietary amount of chondroitin sulfate.

18. A composition as in claim 15 plus a dietary amount of glucosamine plus chondroitin sulfate.

19. A composition as in claim 16 plus a therapeutic amount of a non-steroidal, anti-inflammatory drug.

20. A composition as in claim 17 plus a therapeutic amount of a non-steroidal, anti-inflammatory drug.

21. A composition as in claim 18 plus a dietary amount of glucosamine plus chondroitin sulfate plus a therapeutic amount of a non-steroidal, anti-inflammatory drug.

22. A composition as in claim 1 plus a dietary amount of one or more nutraceutical listed in Table 7.

23. A composition as in claim 22 plus a dietary amount of glucosamine.

24. A composition as in claim 22 plus a dietary amount of chondroitin sulfate.

25. A composition as in claim 23 plus a dietary amount of glucosamine plus chondroitin sulfate.

26. A composition as in claim 23 plus a therapeutic amount of a non-steroidal, anti-inflammatory drug.

27. A composition as in claim 24 plus a therapeutic amount of a non-steroidal, anti-inflammatory drug.

28. A composition as in claim 25 plus a therapeutic amount of a non-steroidal, anti-inflammatory drug.
